(19) United States
(12) Patent Application Publication
(10) Pub. No.: US 2005/0175141 A1
(43) Pub. Date: Aug. 11, 2005

(54) Method for producing tomograms of a periodically moving object with the aid of a focus/detector combination

(76) Inventors: Herbert Bruder, Hoechstadt (DE); Thomas Flohr, Uehlfeld (DE); Karl Stierstorfer, Erlangen (DE)

Correspondence Address: HARNESS, DICKEY & PIERCE, P.L.C., P.O.BOX 8910, RESTON, VA, 20195, US

(21) Appl. No.: 11/038,090

(22) Filed: Jan. 21, 2005

(30) Foreign Application Priority Data
Jan. 22, 2004 (DE) 10 2004 003 367.6

(51) Int. Cl. G21K 1/12; H05G 1/60; G01N 23/00; A61B 6/00

(52) U.S. Cl. 378/8

(57) ABSTRACT

A method and a computed tomograph produce CT images of high resolution by circular scanning of a moving object by scanning sub-segments in a number of successive rest phases of an object under examination, and by respectively reconstructing and reformatting the sub-segments in order subsequently to add up a number of tomograms of the sub-segments. The sum of the sub-segments as a whole reproduce a complementary half segment of a circuit of the focus about the examination object. The moving object is thereby completely scanned, without lateral movement, by the beam being used.

The present application hereby claims priority under 35 U.S.C. §119 on German patent application numbers DE 10 2004 003 367.6 filed Jan. 22, 2004, the entire contents of which is hereby incorporated herein by reference.

### FIELD OF THE INVENTION

The invention generally relates to a method for producing tomograms. In particular, it relates to a method for producing X-ray CT images, of a periodically moving examination object with periodically alternating cyclic phases, if appropriate with movement and rest phases. Preferably, inn order to scan the periodically moving examination object, a focus/detector combination is moved on a circular track about the examination object, detector output signals and movement signals from the examination object simultaneously are measured in order to determine the cyclic phase or the movement and rest phases and stored in a fashion correlated with the detector output signals, and tomograms subsequently are produced with the aid of back-projections by reconstruction and reformatting on the basis of the stored detector output signals.

### BACKGROUND OF THE INVENTION

A computed tomography method for producing tomograms of moving objects is known from laid-open specification DE 199 57 082 A1. Here, in order to display a beating heart in parallel with the scanning process, the movement signals of the heart are recorded by way of an ECG in order thereby to determine the rest phases of the heart and to make exclusive use of images from the rest phase, the X-ray source additionally being active only during the rest phase in the cited document.

Furthermore, reference is made to the publication by T. Flohr, B. Ohnesorge, “Heart-Rate Adaptive Optimization of Spatial and Temporal Resolution for ECG-Gated Multislice Spiral CT of the Heart”, JCAT vol. 25, No. 6, 2001. Algorithms for phase-accurate volume reconstruction of the heart are known from this document for a focus/detector combination moved spirally about the heart in a multirow CT.

The problem of these generally known cardiospiral reconstruction methods resides in the fact that the scanned area has a striped effect owing to the spiral movement of the focus, and so the image quality of the CT records obtained suffers greatly.

### SUMMARY OF THE INVENTION

An object of an embodiment of the invention is therefore to provide a method for producing tomograms of periodically moving examination objects which makes it possible to avoid the striped appearance of the image display.

The inventors have realized that it is possible to adapt the gated AMPR (AMPR=Adaptive Multiplanar Reconstruction) variant, described in German patent application DE 102 07 623 A1, the entire contents of which are hereby incorporated herein by reference, for sequential acquisition of CT data. In the case of a cardio application of an embodiment of the present invention, multilayer projections are measured sequentially in parallel with recording of the patient's ECG in a number of successive cardiac cycles, and image data of the volume of the heart are calculated retrospectively in relation to a selected heart phase, account also being taken of the conical course of the ray.

To reconstruct individual reconstruction layers (pages) it is possible to make use in a known way from the segment to be reconstructed of image stacks, termed segment image stacks or “booklets” in the specialist terminology, of the respective reconstruction segment. The center of the reconstruction segment is determined by a reference projection angle Φ_{ref} that is assigned by way of the ECGs recorded in parallel to a selected heart phase, mostly an area from the rest phase. The minimum length of this reconstruction segment is θ_{scan}≦π. The planes of the reconstruction layers are attached at the reference projection angle to the circular track of the revolving focus, and inclined with reference to an N-row detector such that all the detector data are used in the reconstruction of the M equidistant reconstruction layers (M≧N).

It is possible in general for the reconstruction layers (booklet pages) formed from the image stacks also to be of a curved shape. After the reconstruction of the image stack, it is possible to carry out reformatting in the direction of the system axis with a uniform orientation, corresponding to the target image planes. This can be performed, for example, using a weighting method known per se.

In order to improve the time resolution, the data interval of length θ_{scan} required for the reconstruction can be subdivided into a number of sectors supplementing one another. This is explained below in more detail for the case of a two-segment reconstruction, the data interval of length θ_{scan} being composed of sectors obtained in two successive cardiac cycles. These sectors s₁, s₂ are determined in this case such that they supplement one another in a complementary fashion to form a data interval of length θ_{scan}. In this process, the temporal position in the successive cardiac cycles is to be determined exactly in phase with the aid of the ECG data recorded during the data collection. Segments s₁, s₂ of different lengths are generally yielded thereby.

The time resolution Δt of the determined CT images is dependent here on the local heart rate, and is

$$Δ t = \frac{{θ}_{scan}}{4 π} \cdot {T}_{rot} ,$$

in the most favorable case, in connection with an equal length of the two sectors s₁ and s₂, and

$$Δ t = \frac{{θ}_{scan}}{2 π} \cdot {T}_{rot} ,$$

in the most unfavorable case. In the latter case, one of the two sectors is of length zero.

Successive, reformatted, preferably axial segment image stacks whose assigned reference projection angles are included in the sectors s₁ and s₂ are now determined for each of the sectors s₁ and s₂. The segment images are then added up layer by layer to form a complete CT image.

In the case of a triggered control of the focus, it is also possible to scan only one datastream of length θ_{scan} in accordance with the selected heart phase. A segment image stack is then determined for this datastream in the way specified above. The reconstruction and reformatting are then performed in a way similar to the above described method.

In accordance with the basic ideas outlined above, the inventors propose a method for producing tomograms, in particular X-ray CT images, of an at least partially periodically moving examination object with periodically recurring cyclic phases, if appropriate alternating movement and rest phases, preferably of a heart of a living being, preferably a patient, the method comprising:
in order to scan the examination object, a focus producing a conical beam (conical=formed in the shape of a fan in two mutually perpendicular planes) is moved with a multirow detector, opposite the focus, on a circular track about the examination object, wherein detector output data that represent the attenuation of rays emanating from the focus upon passage through the examination object are collected together with spatial orientation data of the rays, and
the beam is spread out so wide that the volume of the moving examination object is completely covered by circular scanning without additional lateral movement,
at the same time, movement signals, preferably ECG signals, of the examination object are measured for the purpose of detecting the phase, preferably of movement and rest phases, the temporal correlation between the movement data and the detector output data being stored,
subsequently, the detector output signals of individual sub-segments of each detector row, which together each produce a complete segment sweeping at least 180° and represent a rest phase of the moving object, are retrospectively combined,
the complete segments being composed of n sub-segments, preferably n=2, of n successive periods of the moving examination object depending on the desired time resolution per detector row, and
a back-projection with reconstruction and reformatting is carried out with these complete segments.

Thus, in the course of circular movement of the multirow detector data are collected over a number of movement cycles and assembled in a fashion supplementing one another to form a complete data record in a complementary way. Such a data record can then subsequently be calculated using the known reconstruction methods with 2D back-projection methods, and tomograms are produced in a known way. In this case, overall, the time resolution is higher the more movement cycles over which measurement can be performed. However, there are other natural limits in the case of an excessively large number of movement cycles being used—at least when examining patients. Thus, artifacts are produced because of other movements or breathing, or dose problems result because of an excessively long irradiation period. It is therefore mostly more favorable when only two to three movement cycles are added up.

It can be advantageous in principle when considering a moving heart to take data only from the rest phase of the heart, in order to obtain images that are as sharp as possible. However, the ever shorter rotation times of CT also permit concentration to center on an arbitrary cyclic phase of the heart that can also be an interval in an action phase, or even permit a type of “3D image sequence” to be recorded over the complete cardiac cycle.

In order to facilitate the subsequent computation operation, parallel rebinning can be carried out, preferably line by line, before the back-projection in the case of the method according to an embodiment of the invention.

It is advantageous with this method according to an embodiment of the invention when in each case image stacks, generally termed booklets, are formed for a multiplicity (M) of equidistant reconstruction layers from the detector data, in which case the number of the reconstruction layers should be greater than or equal to the number (N) of the detector rows of the multirow detector used, and reformatting is carried out on parallel and equidistant image planes.

It can, furthermore, be advantageous to select the sub-segments of a complete segment to be of different lengths, in which case these sub-segments should, however, supplement one another in a complementary fashion with reference to the scanning angle covered to form a sector sweeping at least 180°, and should lie temporally within the same cyclic phase, preferably an identical interval of a retrospectively determined rest phase with reference to the movement situation of the examination object.

In order to reduce the dose commitment of a patient, the radiation that emanates from the focus can be reduced in a fashion controlled indirectly or directly by the measured movement signals over at least the greater part of the movement phase.

In order to improve the image quality and in order to avoid artifacts at the junctions between the data from different sectors of different cycles, it is favorable for a transitional weighting to be undertaken between the data records when combining the data records.

Furthermore, the data records can be subjected to sinogram weighting in order to prevent image artifacts.

It is also advantageous, if appropriate, on the basis of the existing dependence of the temporal resolution on the cycle period of the movement of the heart and on the rotational speed of the gantry to adapt the rotation frequency of the focus as a function of the measured pulse rate so as to set the theoretically achievable best temporal resolution.

In order to improve the temporal resolution, it can be advantageous in part to carry out the data collection not only over two heart periods, but over three or four heart periods, it being possible for fuzziness to be caused, in turn, by using an excessively large number of heart periods.

It may also further be pointed out in addition that the invention covers both applications with a jointly rotating focus/detector combination and with a rotating focus in conjunction with a cylindrical stationary multirow detector enclosing 2π.

### BRIEF DESCRIPTION OF THE DRAWINGS

The invention is described in more detail below with the aid of an exemplary embodiment and the figures, the following reference symbols being used: 1: CT unit; 2: X-ray tube; 3: Multirow detector; 4: Patient couch; 5: System axis/z-axis; 6: Gantry; 7: Patient; 8: ECG measuring line; 9: Control/Measuring line; 10: Control/Evaluation unit; 11: Display screen; 12: Keyboard; 13: Focus; 14: Beam; 15: Heart; 16: ECG line; 17.x: Section planes; 18: Rest phase; 19: Circular track of the focus; 20.x: Ray planes; 21.x: Parallel rays; 22: Physical detector; 23: R wave; 24: Start of the rest phase; m: Number of the detector rows; n: Number of detector elements per detector row; Θ₁: 1st scanning sector; Θ₂: 2nd complementary scanning sector; Θ₃: 3rd complementary scanning sector; Θ₄: 4th complementary scanning sector.

In the figures:

FIG. 1: shows an illustration of a computed tomograph;

FIG. 2: shows a schematic of a computed tomograph in cross section;

FIG. 3: shows a schematic of a computed tomograph in longitudinal section;

FIG. 4: shows an illustration of the scanning method according to an embodiment of the invention with sectorwise data collection over 2 heart periods;

FIG. 5: shows a schematic of possible data collection over a number of sectors for the purpose of calculating complete CT images, with data collection in 2 sectors of equal length over 2 heart periods;

FIG. 6: shows a schematic of the linewise data combination from two scanning sectors of equal length over 2 heart periods;

FIG. 7: shows a schematic of possible sector combination with data collection in 2 sectors of different length over 2 heart periods;

FIG. 8: shows an illustration of the scanning method according to an embodiment of the invention for the case of sequential scanning with feed in the z-direction;

FIG. 9: shows an illustration of the scanning method according to an embodiment of the invention with sectorwise data collection over 4 heart periods;

FIG. 10: shows a schematic of possible sector combination for complete CT images with data collection in 4 sectors of equal length over 4 periods; and

FIG. 11: shows a schematic of a stack of reconstruction layers in parallel geometry in the case of circular scanning.

### DETAILED DESCRIPTION OF THE EXEMPLARY EMBODIMENTS

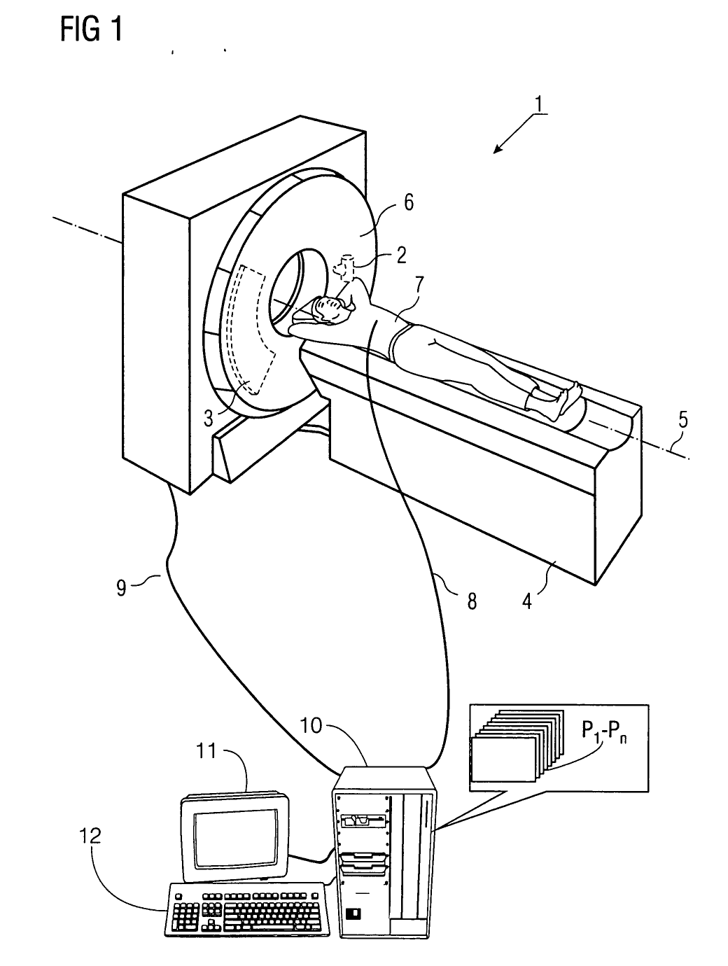

FIG. 1 shows a computed tomograph 1 having a gantry 6 in which a circularly revolving X-ray tube 2 with an opposite multirow detector 3 is located. Also illustrated is a patient 7 who is lying on a patient couch 4 and is moved into the opening of the CT 1 for the scanning operation, there being no relative movement of the patient in the direction of the system axis 5 during the scanning operation, in which the X-ray tube is moved in a circle about the patient. The computed tomograph 1 is controlled by the control and evaluation unit 10 via the control/measuring line 9 over which the data collected by the multirow detector 3 are also transmitted.

Integrated furthermore in the control and evaluation unit 10 is an ECG that measures the potential currents caused by the heart via the ECG measuring line 8 in order to detect the movement situation of the heart at any one time. The control and evaluation unit 10 has internal memories and arithmetic processors via which the programs P₁ to Pₙ for controlling the computed tomograph and for evaluating the collected data are run. Moreover, a keyboard 12 for data input and a monitor 11 for displaying data are connected to the control and evaluation unit.

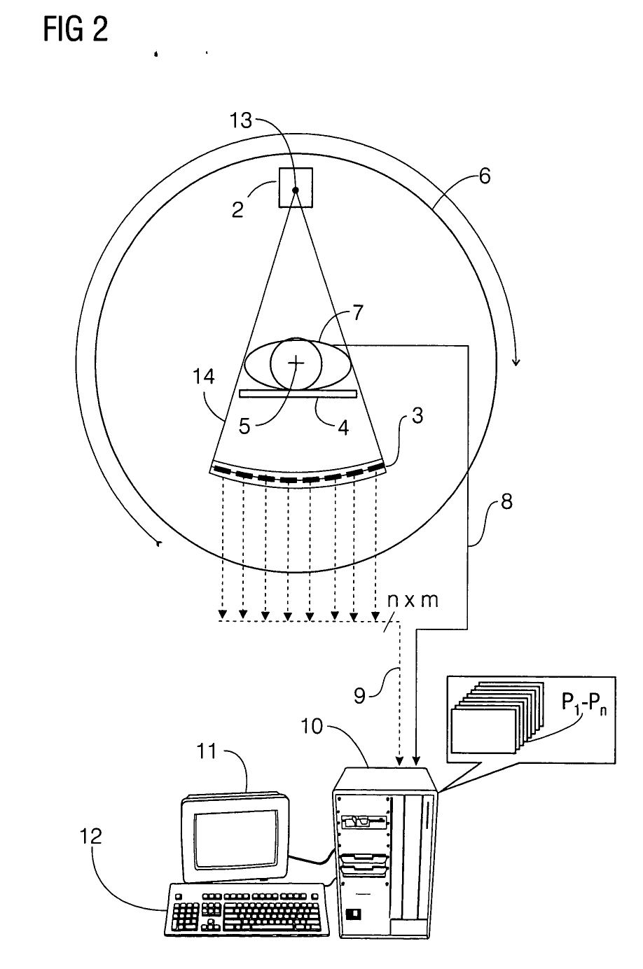

FIG. 2 shows the computed tomograph of FIG. 1 in cross section and in a schematic. Located inside the X-ray tube 2 is a focus 13 from which a beam 14 expanded into a fan emanates and strikes the opposite multi-row detector 3. Upon passage of the X radiation through the patient 7, the X-rays are attenuated differently in accordance with the different trans-irradiated tissue, and the attenuation is measured by the individual detector elements of the detector in an n×m-row matrix, and passed on to the control and evaluation unit 10 via the measuring line 9. According to an embodiment of the invention, position data relating to the current rotary position of the gantry 6 and also the ECG data relating to the ECG measuring line 8 are stored according to an embodiment of the invention in the control and evaluation unit 10 during the measuring operation so that the correlation between the cyclic phase and the detector output data can be accomplished.

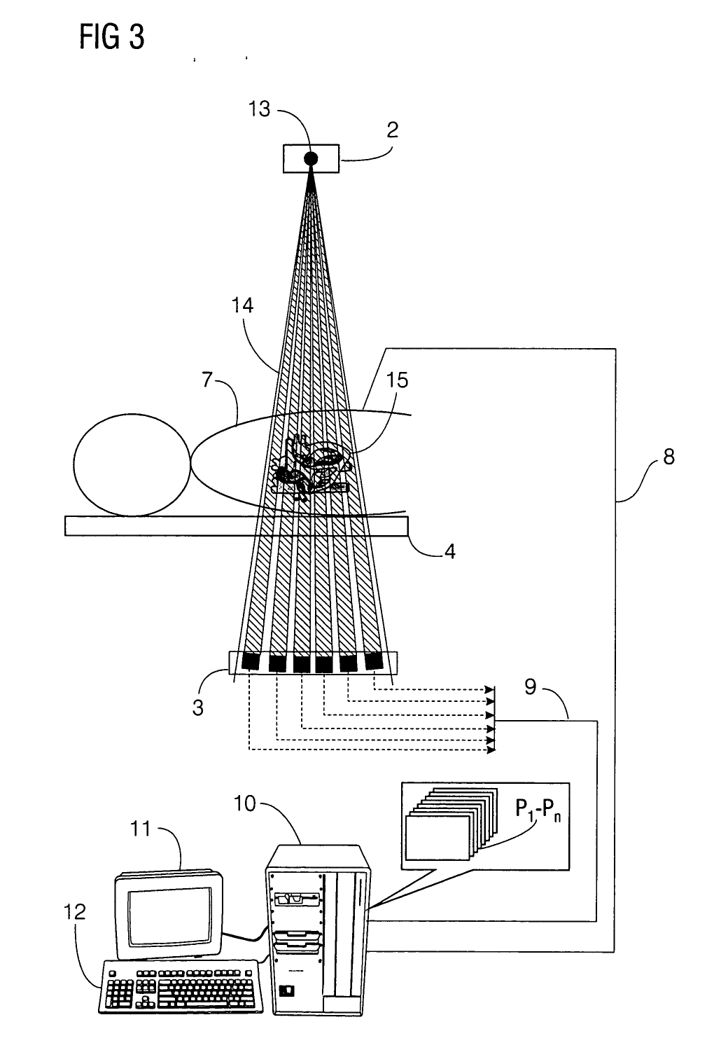

FIG. 3 once again shows the computed tomography unit 1 from FIG. 1, but this time in longitudinal section. Here, the trans-irradiation of a heart 15 beating in the patient 7 is demonstrated schematically. For reasons of clarity, one detector has been illustrated in FIGS. 2 and 3 with a few rows and a few detector elements per row. However, according to an embodiment of the invention these are detectors that have a large number of detector rows and detector elements per detector row so that at least the moving heart can be completely scanned with a single circular scanning operation without simultaneously feeding the patient in the direction of the system axis.

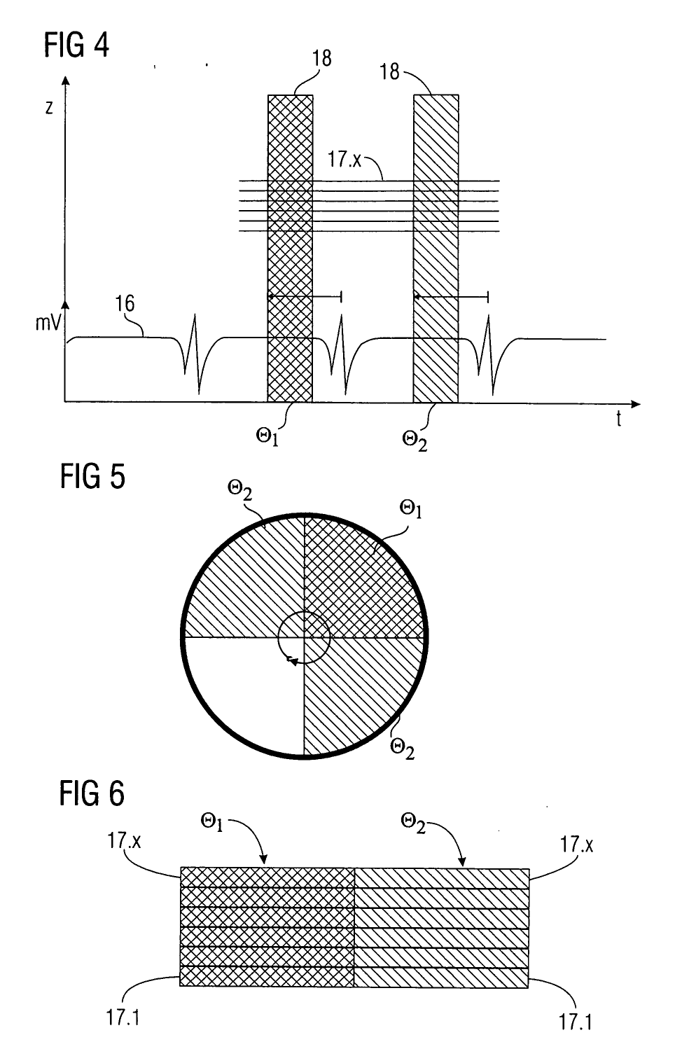

FIG. 4 shows a schematic of the time profile of an inventive circular scanning operation of a heart. Here, the time axis is illustrated on the abscissa, while the ordinate on the one hand shows the system axis or z-axis, and on the other hand shows the measured cardiac activity of the ECG recorder in millivolts (mV).

The ECG line bears the reference symbol 16, the start of the rest phase 24 being determined according to an embodiment of the invention in a retrospective fashion on the basis of the R wave 23. The rest phase itself is illustrated in the bar 18. A number of sequential heartbeat periods are used to evaluate CT images in the section planes 17.x. A total of four heart periods are illustrated in FIG. 4, two juxtaposed heart periods being used with two rest phases 18 for collecting the data.

The sectorwise data collection is illustrated in FIG. 5. Here, the focus or beam traverses a first circle sector Θ₁ during the first rest phase 18, and a second circle sector Θ₂ in the subsequent rest phase 18. Ideally, the rate of rotation of the focus is set in this case such that both sectors each cover 90° and, as illustrated in FIG. 5, supplement one another in a complementary fashion such that overall a complete sector of at least 180° is scanned and the data from the two sectors can be joined to form a complete data record in order therefrom to reconstruct the desired CT images and reformat them axially. Depending on the ratio of rotation time of the focus and the current length of the cardiac cycle, use may be made for this purpose either of the second circle sector Θ₂ lying directly after, or that lying directly in front of, the first circle sector Θ₁. This depends fundamentally in each case on the existing rotation time of the focus and the length of the cardiac cycle.

FIG. 6 shows in accordance with FIGS. 4 and 5 how the data of the multirow detectors obtained from the two sectors Θ₁ and Θ₂ are combined for the further reconstruction. Thus, each row 17.x includes a first portion with data that originate from the first circle sector Θ₁ and a second portion includes data that originate from the second circle sector Θ₂, each circle sector having been acquired in another cardiac cycle.

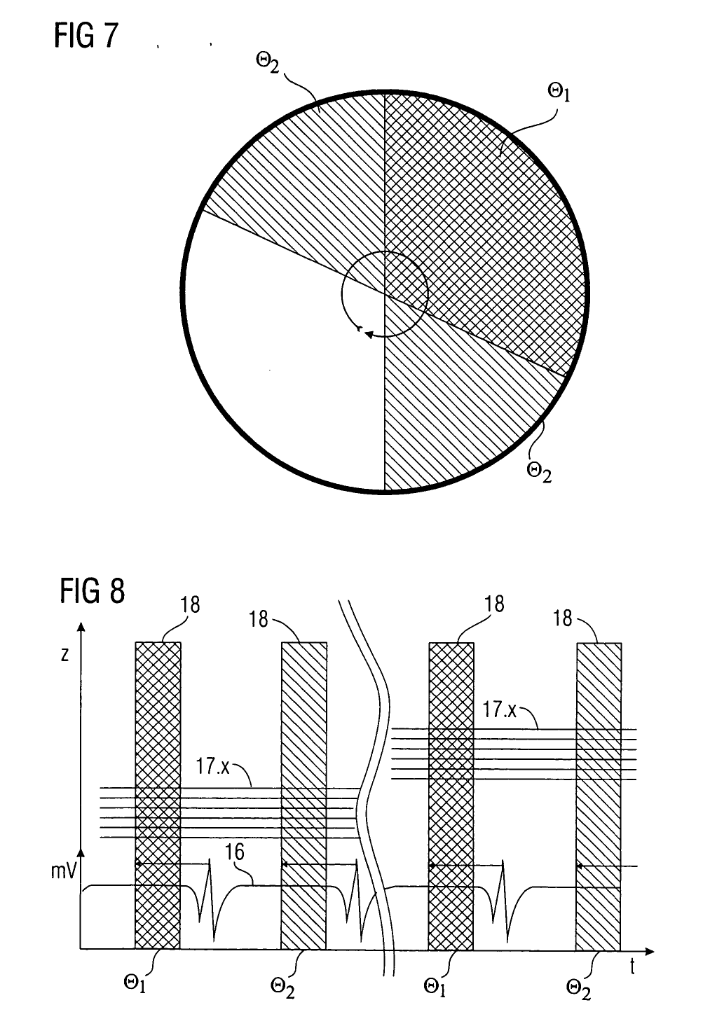

The data collection can be performed in accordance with the situation illustrated in FIG. 7 for the case of non-optimum correspondence between the rotation time of the gantry and the heart rate. Here, the rate of rotation is set relatively high such that the first circle sector Θ₁ sweeps an angle of over 90°. Use is then correspondingly made of an adjacent angle of less than 90° for the second circle sector Θ₂ such that it is possible overall to measure a complete half revolution again and use it for reconstruction.

For the case in which the object to be examined cannot be completely scanned by a single circular scan despite the wide expansion of the scanning beam and the large extent of the multirow detector in the direction of the z-axis, it is also possible for a number of circular scans according to an embodiment of the invention to be juxtaposed sequentially, and to feed in the direction of the system axis between the individual scans. FIG. 8 shows a schematic of such an operation.

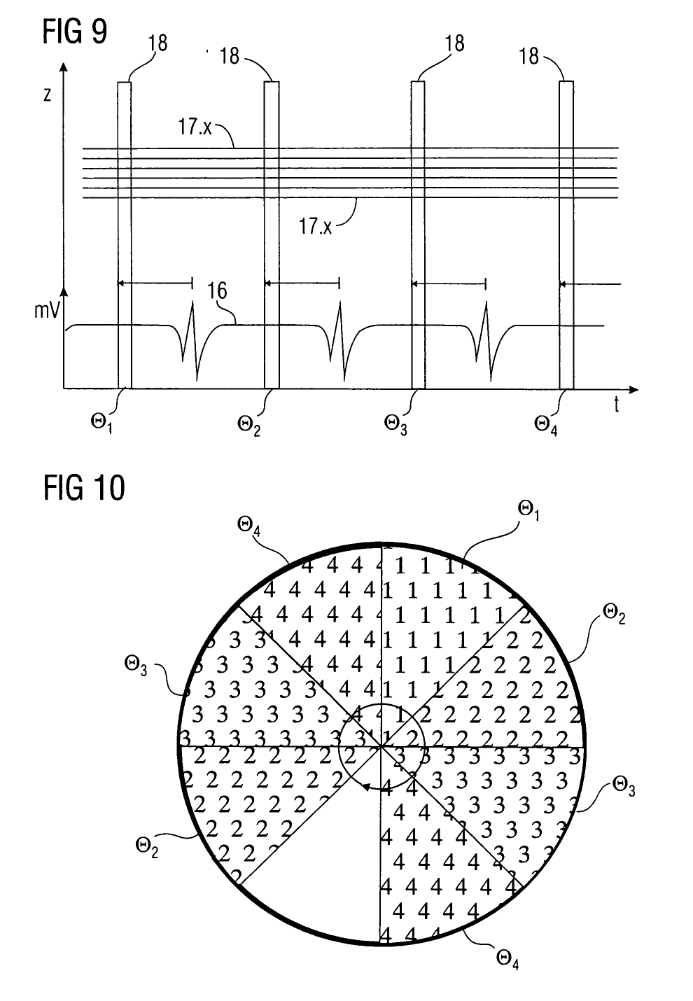

A further increase in the time resolution is illustrated in FIGS. 9 and 10. These figures show scanning over 4 heart periods and 4 circle sectors θ₁-θ₄. In accordance with the multiplication of the scanning sectors, the time span covered within the rest phase is also smaller, and can thereby be fitted even more effectively into a heart phase that is actually motionless, so that the image quality can be substantially improved on the basis of the higher temporal resolution.

FIG. 9 is a schematic of the scanning according to an embodiment of the invention with sector-wise data collection over 4 heart periods, whereas FIG. 10 shows the possible complementary assemblage of sectors that is required in order to obtain complete data records for reconstruction overall. If, with reference to the starting sector—filled up here with “1”—the second sector—filled up with “2”—is measured later by at least 180°, the projection data of this sector are to be reflected by 180° in the correct channel onto the second sector such that sectors arranged successively supplement one another to form 180° overall. The sectors that can be respectively interchanged with one another in a mirror-image fashion are respectively filled up with the same numerals “2”, “3” and “4”. It goes without saying that the example shown illustrates only one possible variant of the data collection with sectors of the same size, other sequences and different sizes of sector likewise being possible.

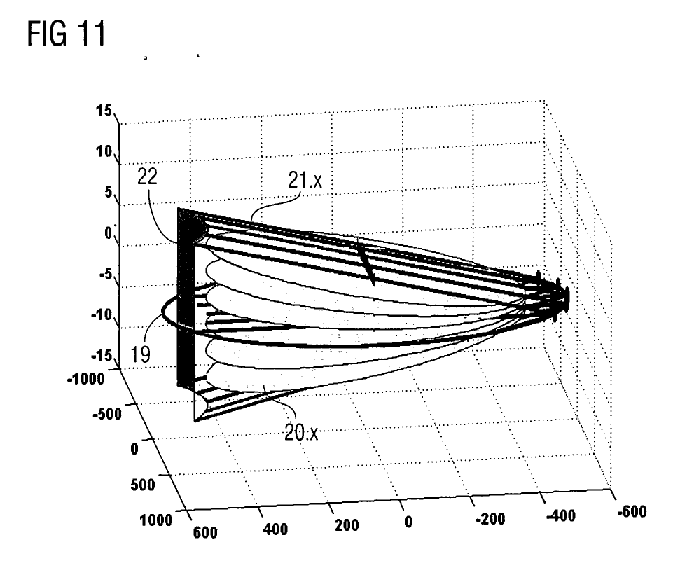

FIG. 11 shows a stack of reconstruction layers in parallel geometry in the case of circular scanning of a sector. Only six fan-shaped reconstruction layers are shown here, as well, for the purpose of a clear illustration. The reconstruction segment has an overall length of π and is assembled from the data of juxtaposed measured data over a number of heart periods. It is also clearly to be seen here that the physical detector 22 is concavely curved in accordance with the parallel rebinning.

As illustrated in FIG. 11, as with all the data collection methods illustrated above the data from each of the scanning sectors are combined from the individual sectors to form a complete π-sector, and in accordance with an embodiment of the invention such fan-shaped image stacks 20.1-20.n are reconstructed and subsequently reformatted in a way known per se to form axial image layers from complete CT images. These axial images then constitute a complete representation of a section of the object under examination.

Thus, overall, the invention represents a method and a computed tomograph in which high-resolution CT images are produced by circular scanning of a moving examination object by virtue of the fact that sub-segments are scanned in a number of successive cyclic phases, and the sub-segments are respectively reconstructed and reformatted per se in order subsequently to add up a number of tomograms of the sub-segments, the sum of the sub-segments reproducing overall a complementary half segment of a circular revolution of the focus about the examination object, the moving examination object being completely scanned by the beam used without lateral movement.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A method for producing tomograms of an at least partially periodically moving examination object with periodically recurring cyclic phases, comprising:
scanning the examination object by moving a focus producing a conical beam in conjunction with a multirow detector, opposite the focus, on a circular track about the examination object, wherein the scanning includes collecting detector output data, representing the attenuation of rays emanating from the focus upon passage through the examination object, together with spatial orientation data of the rays, and wherein the beam is spread out so wide that the volume of the moving examination object is completely covered by circular scanning without additional lateral movement;
measuring at the same time, movement signals of the examination object for the purpose of detecting the cyclic phase, wherein a temporal correlation between the movement data and the detector output data is stored; and
subsequently and retrospectively combining the detector output signals of individual sub-segments of each detector row, which together each produce a complete segment sweeping at least 180° and represent a specific phase sector in the cycle of the moving examination object, wherein the complete segments are composed of n sub-segments of n successive periods of the moving examination object depending on the desired time resolution per detector row, and wherein a back-projection with 2D reconstruction and reformatting may be carried out with the complete segments.

2. The method as claimed in claim 1, wherein the moving heart of a living being is scanned.

3. The method as claimed in claim 2, wherein, as movement signals, ECG signals are measured.

4. The method as claimed in claim 1, wherein parallel rebinning is carried out before the back-projection.

5. The method as claimed in claim 4, wherein the parallel rebinning is carried out line by line.

6. The method as claimed in claim 1, wherein the complete segments are composed of 2 sub-segments from 2 successive periods of the moving examination object.

7. The method as claimed in claim 1, wherein image stacks are respectively formed for M equidistant reconstruction layers from the detector data, wherein M≧N, where N is the number of the detector rows, and reformatting is carried out on parallel and equidistant image planes.

8. The method as claimed in claim 1, wherein the sub-segments of a complete segment are of different lengths, but supplement one another in a complementary fashion with reference to the scanning angle covered, and lie temporally within the same cyclic phase with reference to the movement situation of the examination object.

9. The method as claimed in claim 8, wherein the sub-segments of a complete segment lie within an identical interval of a retrospectively determined rest phase.

10. The method as claimed in claim 1, wherein, when combining the data records, a transitional weighting is undertaken between the data records in order to improve the image quality and to avoid artifacts at the transitions of the data from different sectors of different cycles.

11. The method as claimed in claim 1, wherein the data records are subjected to sinogram weighting in order to prevent image artifacts.

12. The method as claimed in claim 2, wherein, in order to reduce the dose commitment of the examination object, the radiation that emanates from at least one focus is switched off in a fashion controlled indirectly or directly by the measured movement signals over at least the greater part of a movement phase of the heart.

13. The method as claimed in claim 1, wherein the rotation frequency of the focus is set such that at least two sub-segments, that supplement one another to form a complete segment, are swept per cyclic phase considered or per cyclic phase interval considered of the examination object.

14. A computed tomography unit for producing tomograms of an at least partially periodically moving examination object with periodically alternating cyclic phases, comprising:
a focus for scanning the examination object, adapted to produce a conical beam;
a multirow detector located opposite the focus, wherein at least the focus is movably arranged on a circular track about the examination object;
storage means for collecting detector output data that represent the attenuation of rays emanating from the focus upon passage through the examination object, together with spatial orientation data of the rays, wherein the beam is spread out so wide that the volume of the moving examination object can be completely covered by circular scanning without additional lateral movement;
acquisition and storage means for simultaneously collecting movement signals from the examination object in order to detect movement and rest phases, and for storing the temporal correlation between the movement data and detector output data; and
means for retrospectively combining the detector output signals of individual sub-segments of each detector row that together respectively produce a complete segment sweeping at least 180° and represent a specific cyclic phase of the moving examination object, wherein the complete segments are composed of n sub-segments of n successive periods of the moving examination object depending on the desired time resolution per detector row, and wherein a back-projection with 2D reconstruction and reformatting may be carried out with these complete segments.

15. A computed tomography unit comprising program means for carrying out the method of claim 2.

16. The method as claimed in claim 1, wherein the tomograms produced are X-ray CT images.

17. The method as claimed in claim 1, wherein the moving heart of a patient is scanned.

18. The method as claimed in claim 2, wherein, as movement signals, ECG signals for the detection of movement and rest phases are measured.

19. The method as claimed in claim 1, wherein the rotation frequency of the focus is set such that at least two sub-segments, that supplement one another to form a complete segment, are swept per cyclic phase considered or per cyclic phase interval considered, per rest phase and per interval in the rest phase, of the examination object.

20. The method of claim 1, further comprising back-projecting with 2D reconstruction and reformatting with the complete segments.

21. A computed tomography unit for producing tomograms of an at least partially periodically moving examination object with periodically alternating cyclic phases, comprising:
a data collection unit including,
a focus for scanning the examination object,
a multirow detector located opposite the focus, and
a device adapted to collect detector output data that represent the attenuation of rays passing through the examination object, together with spatial orientation data of the rays, wherein a beam of the focus is spread out wide enough to completely cover the volume of the moving examination object by circular scanning, without additional lateral movement;
acquisition and storage device, adapted to simultaneously acquire movement data from the examination object, and to store a temporal correlation between the movement data and the detector output data; and
device for retrospectively combining the detector output signals of individual sub-segments of each detector row, that together respectively produce a complete segment sweeping at least 180° and represent a specific cyclic phase of the moving examination object, wherein the complete segments are composed of n sub-segments of n successive periods of the moving examination object depending on the desired time resolution per detector row, and wherein a back-projection with 2D reconstruction and reformatting may be carried out with these complete segments.
